SEQUENCE LISTING (40 sequences)

SEQ ID NO: 1 - protein, 7 aa, Artificial Sequence
  source: /note=“Description of Artificial Sequence Synthetic peptide”
PKKKRKV

SEQ ID NO: 2 - protein, 7 aa, Artificial Sequence
  source: /note=“Description of Artificial Sequence Synthetic peptide”
PKKKRRV

SEQ ID NO: 3 - protein, 16 aa, Artificial Sequence
  source: /note=“Description of Artificial Sequence Synthetic peptide”
KRPAATKKAGQAKKKK

SEQ ID NO: 4 - protein, 11 aa, Artificial Sequence
  source: /note=“Description of Artificial Sequence Synthetic peptide”
YGRKKRRQRRR

SEQ ID NO: 5 - protein, 9 aa, Artificial Sequence
  source: /note=“Description of Artificial Sequence Synthetic peptide”
RKKRRQRRR

SEQ ID NO: 6 - protein, 9 aa, Artificial Sequence
  source: /note=“Description of Artificial Sequence Synthetic peptide”
PAAKRVKLD

SEQ ID NO: 7 - protein, 11 aa, Artificial Sequence
  source: /note=“Description of Artificial Sequence Synthetic peptide”
RQRRNELKRSP

SEQ ID NO: 8 - protein, 8 aa, Artificial Sequence
  source: /note=“Description of Artificial Sequence Synthetic peptide”
VSRKRPRP

SEQ ID NO: 9 - protein, 8 aa, Artificial Sequence
  source: /note=“Description of Artificial Sequence Synthetic peptide”
PPKKARED

SEQ ID NO: 10 - protein, 8 aa, Artificial Sequence
  source: /note=“Description of Artificial Sequence Synthetic peptide”
PQPKKKPL

SEQ ID NO: 11 - protein, 12 aa, Artificial Sequence
  source: /note=“Description of Artificial Sequence Synthetic peptide”
SALIKKKKKMAP

SEQ ID NO: 12 - protein, 7 aa, Artificial Sequence
  source: /note=“Description of Artificial Sequence Synthetic peptide”
PKQKKRK

SEQ ID NO: 13 - protein, 10 aa, Artificial Sequence
  source: /note=“Description of Artificial Sequence Synthetic peptide”
RKLKKKIKKL

SEQ ID NO: 14 - protein, 10 aa, Artificial Sequence
  source: /note=“Description of Artificial Sequence Synthetic peptide”
REKKKFLKRR

SEQ ID NO: 15 - protein, 20 aa, Artificial Sequence
  source: /note=“Description of Artificial Sequence Synthetic peptide”
KRKGDEVDGVDEVAKKKSKK

SEQ ID NO: 16 - protein, 17 aa, Artificial Sequence
  source: /note=“Description of Artificial Sequence Synthetic peptide”
RKCLQAGMNLEARKTKK

SEQ ID NO: 17 - protein, 38 aa, Artificial Sequence
  source: /note=“Description of Artificial Sequence Synthetic polypeptide”
NQSSNFGPMKGGNFGGRSSGPYGGGGQYFAKPRNQGGY

SEQ ID NO: 18 - protein, 42 aa, Artificial Sequence
  source: /note=“Description of Artificial Sequence Synthetic polypeptide”
RMRIZFKNKGKDTAELRRRRVEVSVELRKAKKDEQILKRRNV

SEQ ID NO: 19 - protein, 20 aa, Artificial Sequence
  source: /note=“Description of Artificial Sequence Synthetic peptide”
GRKKRRQRRRPPQPKKKRKV

SEQ ID NO: 20 - protein, 19 aa, Artificial Sequence
  source: /note=“Description of Artificial Sequence Synthetic peptide”
PLSSIFSRIGDPPKKKRKV

SEQ ID NO: 21 - protein, 24 aa, Artificial Sequence
  source: /note=“Description of Artificial Sequence Synthetic peptide”
GALFLGWLGAAGSTMGAPKKKRKV

SEQ ID NO: 22 - protein, 27 aa, Artificial Sequence
  source: /note=“Description of Artificial Sequence Synthetic peptide”
GALFLGFLGAAGSTMGAWSQPKKKRKV

SEQ ID NO: 23 - protein, 21 aa, Artificial Sequence
  source: /note=“Description of Artificial Sequence Synthetic peptide”
KETWWETWWTEWSQPKKKRKV

SEQ ID NO: 24 - protein, 11 aa, Artificial Sequence
  source: /note=“Description of Artificial Sequence Synthetic peptide”
YARAAARQARA

SEQ ID NO: 25 - protein, 11 aa, Artificial Sequence
  source: /note=“Description of Artificial Sequence Synthetic peptide”
THRLPRRRRRR

SEQ ID NO: 26 - protein, 11 aa, Artificial Sequence
  source: /note=“Description of Artificial Sequence Synthetic peptide”
GGRRARRRRRR

SEQ ID NO: 27 - protein, 12 aa, Artificial Sequence
  source: /note=“Description of Artificial Sequence Synthetic peptide”
RRQRRTSKLMKR

SEQ ID NO: 28 - protein, 27 aa, Artificial Sequence
  source: /note=“Description of Artificial Sequence Synthetic peptide”
GWTLNSAGYLLGKINLKALAALAKKIL

SEQ ID NO: 29 - protein, 33 aa, Artificial Sequence
  source: /note=“Description of Artificial Sequence Synthetic polypeptide”
KALAWEAKLAKALAKALAKHLAKALAKALKCEA

SEQ ID NO: 30 - protein, 16 aa, Artificial Sequence
  source: /note=“Description of Artificial Sequence Synthetic peptide”
RQIKIWFQNRRMKWKK

SEQ ID NO: 31 - DNA, 17062 bp, Artificial Sequence
  source: /note=“Description of Artificial Sequence Synthetic polynucleotide”
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgttctagccgccaccatggacaagaagtacagcatcggcctggacatcggcaccaactctgtgggctgggccgtgatcaccgacgactacaaggtgcccagcaagaaattcaaggtgctgggcaacaccgaccggcacagcatcaagaagaacctgatcggcgccctgctgttcggctctggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaagctggccgacagcaccgacaaggccgacctgagactgatctacctggccctggcccacatgatcaagttccggggccacttcctgatcgagggcgacctgaaccccgacaacagcgacgtggacaagctgttcatccagctggtgcagatctacaatcagctgttcgaggaaaaccccatcaacgccagcagagtggacgccaaggccatcctgagcgccagactgagcaagagcagacggctggaaaatctgatcgcccagctgcccggcgagaagcggaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacagcgagatcaccaaggcccccctgtccgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttcagaatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgcggaagcccgcctttctgagcggcgagcagaaaaaggccatcgtggacctgctgttcaagaccaaccggaaagtgaccgtgaagcagctgaaagaggactacttcaagaaaatcgagtgcttcgacagcgtggaaatcagcggcgtggaagatcggttcaacgcctccctgggcgcctatcacgatctgctgaaaattatcaaggacaaggacttcctggacaatgaggaaaacgaggacattctggaagatatcgtgctgaccctgacactgtttgaggaccggggcatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggacactctctgcacgagcagatcgccaatctggccggatcccccgccattaagaagggcatcctgcagacagtgaagattgtggacgagctcgtgaaagtgatgggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacattgtgccccagtccttcatcaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcattaagcggcagctggtggaaacccggcagatcacaaagcacgtggcacagatcctggactcccggatgaacactaagtacgacgagaacgacaaactgatccgggaagtgaaagtgatcaccctgaagtccaagctggtgtccgacttcagaaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgattacaaggtgtacgacgtgcggaagatgatcgccaagagcgagcaggaaatcggcaaggctaccgccaagtacttcttctacagcaacatcatgaactttttcaagaccgagatcacactggccaacggcgagatcagaaagcggcctctgatcgagacaaacggcgaaaccggggagatcgtgtgggataagggccgggattttgccacagtgcggaaagtgctgtccatgccccaagtgaatatcgtgaaaaagaccgaggtgcagaccggcggcttcagcaaagagtctatcctgcccaagaggaactccgacaagctgatcgccagaaagaaggattgggaccctaagaagtacggcggctttgacagccccaccgtggcctactctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagcggatgctggcttctgccggcgaactgcagaagggaaacgagctggccctgccctccaaatatgtgaacttcctgtacctggccagccactatgagaagctgaagggctcccccgaggataatgagcagaaacagctgtttgtggaacagcacaagcactacctggacgagatcatcgagcagattagcgagttctccaagcgcgtgatcctggccgatgccaacctggacaaggtgctgagcgcctacaacaagcaccgggataagcccatcagagagcaggccgagaatatcatccacctgtttaccctgaccaacctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgaccccaagaaaaagcgcaaagtggttaacggcagtggagagggcagaggaagtctgctaacatgcggtgacgtcgaggagaatcctggcccacgatcgatgcatagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggctgttaactgagcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgagcaagatctacatcgacgagcggagcaacgccgagattgtgtgcgaggccatcaagaccatcggaatcgaaggcgccacagccgctcagctgaccagacagctgaacatggaaaagcgggaagtgaacaaggccctgtacgacctgcagagaagcgccatggtgtacagcagcgacgacatccctcctcggtggtttatgaccacagaggccgacaagcctgacgccgatgctatggccgacgtgatcatcgacgacgtgtcccgcgagaagtccatgagagaggaccacaagagcttcgacgatgtgatccccgccaagaagatcatcgattggaagggcgccaatcctgtgaccgtgatcaacgagtactgccagatcaccagaagagactggtccttccggatcgagagcgtgggccctagcaatagccctaccttctacgcctgcgtggacatcgacggcagagtgttcgataaggccgacggcaagagcaagcgggacgccaaaaacaatgccgccaagctggccgtggataagctgctgggctatgtgatcatccggttctaaggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgaaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaattaatctagaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctctaatacgactcactatag

SEQ ID NO: 32 - DNA, 17065 bp, Artificial Sequence
  source: /note=“Description of Artificial Sequence Synthetic polynucleotide”
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgttctagccgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggcggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgcgctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcgcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgaccccaagaaaaagcgcaaagtggttaacggcagtggagagggcagaggaagtctgctaacatgcggtgacgtcgaggagaatcctggcccacgatcgatgcatagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggctgttaactgagcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgagcaagatctacatcgacgagcggagcaacgccgagattgtgtgcgaggccatcaagaccatcggaatcgaaggcgccacagccgctcagctgaccagacagctgaacatggaaaagcgggaagtgaacaaggccctgtacgacctgcagagaagcgccatggtgtacagcagcgacgacatccctcctcggtggtttatgaccacagaggccgacaagcctgacgccgatgctatggccgacgtgatcatcgacgacgtgtcccgcgagaagtccatgagagaggaccacaagagcttcgacgatgtgatccccgccaagaagatcatcgattggaagggcgccaatcctgtgaccgtgatcaacgagtactgccagatcaccagaagagactggtccttccggatcgagagcgtgggccctagcaatagccctaccttctacgcctgcgtggacatcgacggcagagtgttcgataaggccgacggcaagagcaagcgggacgccaaaaacaatgccgccaagctggccgtggataagctgctgggctatgtgatcatccggttctaaggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgaaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaattaatctagaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctctaatacgactcactatag

SEQ ID NO: 33 - DNA, 16264 bp, Artificial Sequence
  source: /note=“Description of Artificial Sequence Synthetic polynucleotide”
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgttctagccgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggcggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgcgctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcgcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgaccccaagaaaaagcgcaaagtgtgagcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgagcaagatctacatcgacgagcggagcaacgccgagattgtgtgcgaggccatcaagaccatcggaatcgaaggcgccacagccgctcagctgaccagacagctgaacatggaaaagcgggaagtgaacaaggccctgtacgacctgcagagaagcgccatggtgtacagcagcgacgacatccctcctcggtggtttatgaccacagaggccgacaagcctgacgccgatgctatggccgacgtgatcatcgacgacgtgtcccgcgagaagtccatgagagaggaccacaagagcttcgacgatgtgatccccgccaagaagatcatcgattggaagggcgccaatcctgtgaccgtgatcaacgagtactgccagatcaccagaagagactggtccttccggatcgagagcgtgggccctagcaatagccctaccttctacgcctgcgtggacatcgacggcagagtgttcgataaggccgacggcaagagcaagcgggacgccaaaaacaatgccgccaagctggccgtggataagctgctgggctatgtgatcatccggttctaaggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgaaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaattaatctagaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctctaatacgactcactatag

SEQ ID NO: 34 - DNA, 22 bp, Artificial Sequence
  source: /note=“Description of Artificial Sequence Synthetic oligonucleotide”
tagttggagctggtggcgtagg

SEQ ID NO: 35 - DNA, 23 bp, Artificial Sequence
  source: /note=“Description of Artificial Sequence Synthetic oligonucleotide”
gagtccgagcagaagaagaaggg

SEQ ID NO: 36 - DNA, 22 bp, Artificial Sequence
  source: /note=“Description of Artificial Sequence Synthetic primer”
gatacacgtctgcagtcaactg

SEQ ID NO: 37 - DNA, 21 bp, Artificial Sequence
  source: /note=“Description of Artificial Sequence Synthetic primer”
gcatattactggtgcaggacc

SEQ ID NO: 38 - DNA, 20 bp, Artificial Sequence
  source: /note=“Description of Artificial Sequence Synthetic primer”
gcctgagtgttgaggcccca

SEQ ID NO: 39 - DNA, 20 bp, Artificial Sequence
  source: /note=“Description of Artificial Sequence Synthetic primer”
gtccctctgtcaatggcggc

SEQ ID NO: 40 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2022/0186235 A1
(43) Pub. Date: Jun. 16, 2022

(54) SYNTHETIC SELF-REPLICATING RNA VECTORS ENCODING CRISPR PROTEINS AND USES THEREOF

(71) Applicant: EMD Millipore Corporation, Burlington, MA (US)

(72) Inventor: Naohisa Yoshioka, Wildomar, CA (US)

(21) Appl. No.: 17/606,644

(22) PCT Filed: May 13, 2020
(86) PCT No.: PCT/US2020/032688

§ 371 Date: Oct. 26, 2021

Related U.S. Application Data
(60) Provisional application No. 62/847,032, filed on May 13, 2019.

(51) Int. Cl. C12N 15/79; C12N 15/10; C12N 9/22; C12N 15/11

(52) U.S. Cl. CPC C12N 15/79; C12N 15/102; C12N 2310/20; C12N 15/111; C12N 9/22

(57) ABSTRACT

Synthetic, noninfectious, self-replicating RNA vectors that encode CRISPR proteins are provided. Each self-replicating RNA vector comprises a sequence encoding a plurality of non-structural replication complex proteins from an alphavirus and a sequence encoding a CRISPR protein. Also provided are methods for genome editing in which a synthetic self-replicating RNA vector is transfected into cells along with at least one corresponding guide RNA.

### RELATED APPLICATIONS

The present application claims the benefit of priority of U.S. Provisional Patent Application No. 62/847,032, filing date May 13, 2019, the entire content of which is incorporated herein in its entirety.

### SEQUENCE LISTING

This application contains a Sequence Listing that has been submitted in ASCII format via EFS-Web and is hereby incorporated by reference in its entirety. The ASCII copy, created on May 13, 2020, is named P19-084_WO-PCT_SL.txt, and is 77,524 bytes in size.

### FIELD

The present disclosure relates to synthetic self-replicating RNA vectors that encode CRISPR proteins, wherein the synthetic self-replicating RNA vectors can be transfected into cells along with corresponding guide RNAs for genome editing methods.

### BACKGROUND

The recent development of the Clustered Regularly Interspaced Short Palindromic Repeats (CRISPR) and CRISPR-associated (Cas) CRISPR/Cas systems as genome editing tools has provided unprecedented ease and simplicity to engineer site-specific endonucleases for eukaryotic genome modification. Many delivery systems have been developed to deliver CRISPR to eukaryotic cells. However, lentiviral, retroviral, and plasmid systems carry the risk of integration of the CRISPR coding sequence into the host cell genome. There is a need, therefore, for a CRISPR gene delivery system that provides robust expression of CRISPR proteins and avoids the risk of genome integration.

### SUMMARY

Among the various aspects of the present disclosure is the provision of self-replicating RNA vectors encoding CRISPR proteins.

One aspect of the disclosure provides self-replicating RNA vectors comprising sequence encoding a plurality of non-structural replication complex proteins from an alphavirus and sequence encoding a CRISPR protein. The CRISPR protein can be a type II Cas9 protein, a type V Cas12 protein, a type VI Cas13 protein, a CasX protein, or a CasY protein. In certain embodiments, the CRISPR protein can be Streptococcus pyogenes Cas9, Francisella novicida Cas9, Staphylococcus aureus Cas9, Streptococcus thermophilus Cas9, Streptococcus pasteurianus Cas9, Campylobacter jejuni Cas9, Neisseria meningitis Cas9, Neisseria cinerea Cas9, Francisella novicida Cas12, Acidaminococcus sp. Cas12, Lachnospiraceae bacterium ND2006 Cas12, Leptotrichia wadei Cas13a, Leptotrichia shahii Cas13a, Prevotella sp. P5-125 Cas13, or Ruminococcus flavefaciens Cas13d. In specific iterations, the CRISPR protein is Streptococcus pyogenes Cas9 or Staphylococcus aureus Cas9.

In some situations, the sequence encoding the CRISPR protein can comprise at least one nucleotide insertion, deletion, and/or substitution such that the CRISPR protein has altered catalytic activity, improved target site specificity, and/or decreased off-target effects. The CRISPR protein can have double-stranded cleavage activity, can cleave one strand of double-stranded sequence, or can be devoid of all cleavage activity.

The CRISPR protein also can be linked to at least one heterologous domain. For example, the CRISPR protein can be linked to at least one nuclear localization signal. The CRISPR protein also can be linked to at least one fluorescent protein, at least one chromatin modulating motif, at least one epigenetic modification domain, at least one transcriptional regulation domain, at least one RNA aptamer binding domain, or combinations thereof.

The sequence of the self-replicating RNA vector encoding the plurality of non-structural replication complex proteins from an alphavirus can be derived from Aura virus, Babanki virus, Barmah Forest virus, Bebaru virus, Buggy Creek virus, Chikungunya virus, Eastern equine encephalitis virus, Everglades virus, Fort Morgan virus, Getah virus, Highlands J virus, Kyzylagach virus, Mayaro virus, Middelburg virus, Mucambo virus, Ndumu virus Pixuna virus, O'nyong-nyong virus, Ross River virus, Sagiyama virus, Semliki Forest virus, Sindbis virus, Una virus, Venezuelan equine encephalitis virus, Western equine encephalitis virus, or Whataroa virus. These various sequences and derivatives thereof are known and can be found in the scientific literature, which is hereby incorporated by reference herein in their entirety. In specific embodiments, the sequence encoding the plurality of non-structural replication complex proteins is from Venezuelan equine encephalitis virus. See, e.g., Yoshioka et al. (Cell Stem Cell 13, 246-254, Aug. 1, 2013) and/or Petrakova et al. (J. Virology; vol. 72, no. 12, June 2005, p. 7597-7608) each of which is hereby incorporated by reference herein in their entirety. The self-replicating RNA vector can further comprise sequence encoding a selectable marker and/or sequence encoding a interferons response inhibitor.

Another aspect of the present disclosure provides complexes comprising the self-replicating RNA vectors described above and at least one guide RNA engineered to complex with the CRISPR protein coded by the self-replicating RNA vector.

The present disclosure also provides eukaryotic cells or cell lines comprising the self-replicating RNA vectors disclosed herein.

A further aspect of the present disclosure encompasses plasmid vectors encoding the self-replicating RNA vectors.

Still another aspect of the present disclosure provides methods for targeted genome editing. The methods comprise introducing into eukaryotic cells one of the self-replicating RNA vectors disclosed herein and at least one guide RNA that is engineered to complex with the CRISPR protein coded by the self-replicating RNA vector.

Other aspects and iterations of the present disclosure are described in more detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A presents schemes showing the structure of the Simplicon™ cloning vector, and each VEE-Cas9 RNA described herein.

FIG. 1B shows agarose gel electrophoresis of VEE-Cas9 RNAs. Lane 1: RNA markers (200-6000 bases); Lane 2: eSpCas9 RNA; Lane 3: eSp-Cas9-GFP RNA; Lane 4: Cas9-GFP RNA.

FIG. 1C presents images of GFP(TagGFP2) expression in VEE-Cas9 transfected cells. VEE-Cas9 RNA and B18R-E3L RNA were co-transfected into HFFs on day 1, and GFP expression was captured with fluorescence microscope on day 2.

FIG. 1D presents a Western blot of Cas9 proteins. Lane 1: control; Lane 2: eSpCas9; Lanes 3&4: eSpCas9-GFP2; Lanes 5&6: Cas9-GFP2.

FIG. 2A shows images of GFP expression on day 2 after HFFs were co-transfected simultaneously or sequentially with VEE-Cas9-TagGFP2 and synthetic 1-piece or 2-piece gRNAs targeted to K-Ras.

FIG. 2B presents efficiency of genome editing following co-transfection or sequential transfection with VEE-Cas9 RNA and gRNA. Cells were collected on day 3 for co-transfection (simultaneous), day 4 for sequential transfection, and day 6 (after completing of puromycin selection). Indels were detected with Guide-iT Mutation Detection kit. +/− indicates resolvase treatment. The efficiency (%) of cleavage is presented below the gel images.

FIG. 2C shows images of GFP expression of human iPSC cell lines on day 2 following sequential transfection with VEE-Cas9-TagGFP2 and 1-piece of K-Ras gRNA (25 nM).

FIG. 2D presents efficiency of genome editing in human iPSCs shown in FIG. 2C. Cells were collected on day 5 (3 days after gRNA transfection) and day 7 (after completing of puromycin selection). Indels were detected with Guide-iT Mutation Detection kit. +/− indicates resolvase treatment. The efficiency (%) of cleavage is presented below the gel images.

FIG. 3A shows efficiency of genome editing in HFFs transfected with VEE-Cas9 (S-Cas9) and B18R-E3L RNA or infected with Lentivirus Cas9 (LV-Cas9), and then selected with puromycin (0.8 μg/mL) or blasticidin (4 μg/mL), respectively, for 7 days. Cells were passaged on day 7 and transfected on day 9 with 1-piece of K-Ras or EMX-1 sgRNA. Cells were collected on day 11 and analyzed for genome editing. +/− indicated resolvase treatment. % shows the efficiency for cleavage.

FIG. 3B presents Guava flow cytometry analysis of GFP expression on day 4 of HEK293T cells co-transfected with Cas9 plasmid and K-Ras gRNA plasmid, or co-transfected with VEE-Cas9-TagGFP2 RNA, B18R-E3L RNA, and 1-piece of K-Ras-gRNA.

FIG. 3C shows efficiency of genome editing in the cells described in FIG. 3B. +/− indicated resolvase treatment. % shows the efficiency for cleavage.

FIG. 4A presents GFP expression as analyzed with Guava flow cytometry in HFF cells that were generated by puromycin selection for a month after transfection with VEE-Cas9-TagGFP2, and genome editing after co-transfection with 1-piece K-Ras sgRNA.

FIG. 4B shows GFP expression as analyzed with Guava flow cytometry in HEK293T cells that were generated by puromycin selection for a month after transfection with VEE-Cas9-TagGFP2, and genome editing after co-transfection with 1-piece EMX-1 sgRNA.

FIG. 4C compares genome editing in HFFs sequentially transfected with VEE-eSpCas9, VEE-eSpCas9-TagGFP2, or VEE-Cas9-TagGFP2, and 1-piece EMX-1 gRNA. Cells were collected on day 5 and analyzed for genome editing.

FIG. 4D compares genome editing in HEK293T cells sequentially transfected with VEE-Cas9-TagGFP2 or VEE-eSpCas9-TagGFP2, B18R-E3L RNA, and 1-piece EMX-1 gRNA. Cells were collected on day 4 and analyzed for genome editing.

FIG. 5A presents images of GFP or RFP expression in VEE-Cas9-D10A-TagGFP2 and VEE-Cas9-D10A-TagRFP transfected HEK293T cells on day 1 and day, respectively.

FIG. 5B presents images of GFP or RFP expression after puromycin selection on day 14. The GFP expression was also analyzed with Guava flow cytometry.

FIG. 5C compares genome editing in HEK 293T cells sequentially transfected with VEE-Cas9-D10A-TagGFP2, VEE-Cas9-D10A-TagRFP, or VEE-Cas9-TagGFP2 on day 1 and EMX-1 sgRNA-1, 9, or both on day 2. Cells were collected on day 5 and analyzed for genome editing.

FIG. 5D compares genome editing in HEK 293T cell lines expressing VEE-Cas9-D10A-TagGFP2, VEE-Cas9-D10A-TagRFP, or VEE-Cas9-TagGFP2. Each cell lines were transfected with EMX-1 sgRNA-1 and/or -9 on day1. Cells were collected on day 4 and analyzed for genome editing.

FIG. 6A shows the Rab11-BamHI oligo insertion by the cleavage of Cas9 in pooled U2OS or HEK293T cells as indicated. U2OS or HEK 293T cells were sequentially transfected with VEE-Cas9-TagGFP2 or VEE-Cas9-D10A-GFP2 on day 1, and the sgRNA(s) and the Rab11-BamHI oligo on day 2. Cells were collected on day 5 and analyzed for BamHI insertion. The gel images show the BamHI digestion generated by the Rab-11-BamHI oligo insertion at the cleavage site of VEE-Cas9-TagGFP2 (left) or VEE-Cas9-D10A-GFP2 (right).

FIG. 6B shows the Rab11-BamHI oligo insertion at the cleavage of Cas9 in isolated clones. U2OS or human iPSCs were sequentially transfected with VEE-Cas9-GFP2 on day 1, and the sgRNA(s) and the Rab11-BamHI oligo on day 2. Cells were selected with puromycin and clones were isolated for analysis. The gel images show the BamHI digestion generated by the Rab-11-BamHI oligo insertion at the cleavage site of VEE-Cas9-TagGFP2.

FIG. 6C shows the insertion of the GFP fragment at the cleavage site of Cas9. HEK293 cells were sequentially transfected with VEE-Cas9-RFP on day 1, and sgRNA for GAPDH location and GFP fragment on day 2. GFP expression was captured on day 5.

### DETAILED DESCRIPTION

The present disclosure provides synthetic, noninfectious, self-replicating RNA vectors based on alphaviruses in which sequences encoding the structural viral proteins has been removed and replaced with sequence encoding a CRISPR protein. The self-replicating RNA vectors are single-stranded RNAs that mimic cellular mRNAs with 5′ caps and poly(A) tails. In certain embodiments, the self-replicating RNA vectors do not utilize DNA intermediates, and as a consequence, there is no risk for genomic integration of the CRISPR sequence. The self-replicating RNA vectors allow for robust expression of the CRISPR protein over multiple cell generations. Co-transfection of cells with a corresponding guide RNA permits targeted genome editing. The levels of CRISPR expression diminish over time due to dilution and degradation. Also provided herein are methods of using the self-replicating RNA vectors to introduce CRISPR proteins into cells in conjunction with corresponding guide RNA for genome editing.

### (I) RNA Vectors Encoding CRISPR Proteins

One aspect of the present disclosure provides synthetic self-replicating RNA vectors that encode CRISPR proteins. The self-replicating RNA vectors comprise sequences that ensure replication of the RNA vector over several cell generations, as well as translation of heterologous protein sequences (e.g., at least one CRISPR protein). The self-replicating RNA vectors are based on modified alphaviruses that encode a plurality of replication complex proteins, but in which the viral structural genes have been removed and replaced with sequence encoding at least one CRISPR protein. Thus, upon entry into a cell, the RNA serves as a template for translation of the viral replication complex proteins and the CRISPR protein(s). The viral replication complex proteins form replication complexes, which allow for further replication of the RNA vector in the cytoplasm of the cell. The replicated RNA cannot recombine with cellular DNA, and thus, there is no risk of integrating CRISPR sequences into the genome of the cell.

(a) Synthetic Self-Replicating RNA

The synthetic self-replicating RNA (or replicon) contains all the sequence elements needed for translation of the encoded proteins and replication of the RNA vector. In particular, the replicon is based on a modified alphavirus in which the non-structural replicase genes are maintained and the structural genes (needed to make an infectious particle) are removed. In various embodiments, the modified alphavirus can be derived from Aura virus, Babanki virus, Barmah Forest virus, Bebaru virus, Buggy Creek virus, Chikungunya virus, Eastern equine encephalitis virus, Everglades virus, Fort Morgan virus, Getah virus, Highlands J virus, Kyzylagach virus, Mayaro virus, Middelburg virus, Mucambo virus, Ndumu virus Pixuna virus, O'nyong-nyong virus, Ross River virus, Sagiyama virus, Semliki Forest virus, Sindbis virus, Una virus, Venezuelan equine encephalitis virus, Western equine encephalitis virus, or Whataroa virus. These various sequences and derivatives thereof are known and can be found in the scientific literature, which is hereby incorporated by reference herein in their entirety. In specific embodiments, the synthetic self-replicating RNA is based on a modified Venezuelan equine encephalitis (VEE) virus, in which the structural genes have been removed. See, e.g., Yoshioka et al. (Cell Stem Cell 13, 246-254, Aug. 1, 2013) and/or Petrakova et al. (J. Virology; vol. 72, no. 12, June 2005, p. 7597-7608) each of which is hereby incorporated by reference herein in their entirety.

The self-replicating RNA comprises a sequence encoding a plurality of non-structural replication complex proteins. In specific embodiments, the synthetic self-replicating RNA can encode four non-structural replication complex proteins (i.e., nsP1, nsP2, nsP3, nsP4). The non-structural replication complex proteins can be encoded by a single open reading frame (ORF).

The self-replicating RNA vector further comprises sequence encoding at least one CRISPR protein, which are detailed below in section (I)(b).

In general, the self-replicating RNA vector comprises a 5′ cap, a 5′ untranslated region (UTR) at the 5′ end and a 3′ UTR and a poly A tail at the 3′ end. The self-replicating RNA vector generally comprises a promoter upstream of the sequence encoding the CRISPR protein. The upstream promoter can be a 26S subgenomic promoter.

In some embodiments, the self-replicating RNA vector can further comprise sequence coding at least one selectable marker and/or sequence encoding an inhibitor of an interferon response. Non-limiting examples of suitable selectable marker include puromycin, geneticin, neomycin, hydromycin B, blastidinin S, and the like. Examples of suitable interferon response inhibitors include, without limit, vaccinia virus protein E3L, vaccinia virus protein B18R, influenza virus protein NS1, or lymphocytic choriomeningitis virus nucleoprotein.

The various protein coding sequences can be separated by internal ribosome entry sequences (IRES) or sequences encoding 2A peptides. Non-limiting examples of suitable 2A peptides include the thosea asigna virus 2A peptide or T2A, foot-and-mouth disease virus 2A peptide or F2A, equine rhinitis A virus 2A peptide or E2A, and porcine teschovirus-1 2A peptide or P2A.

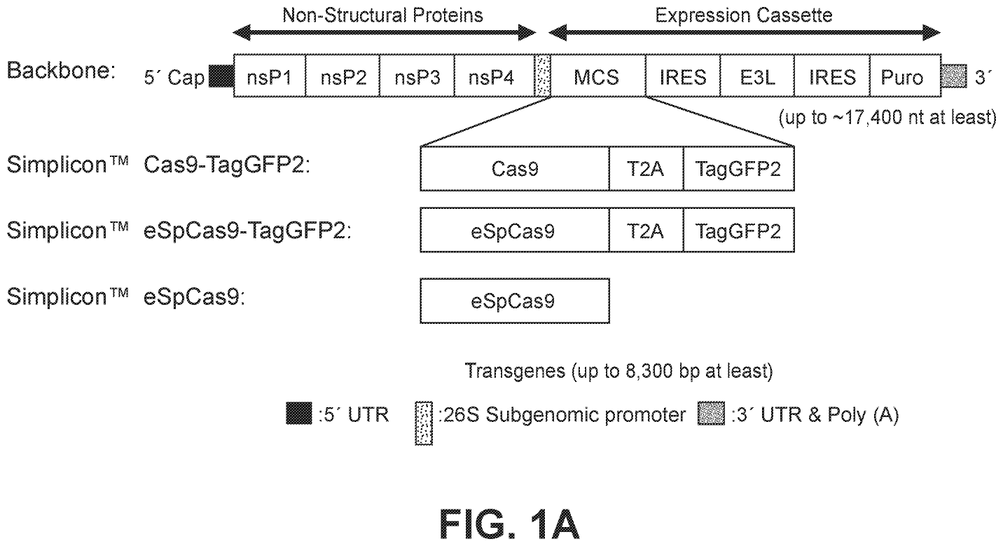

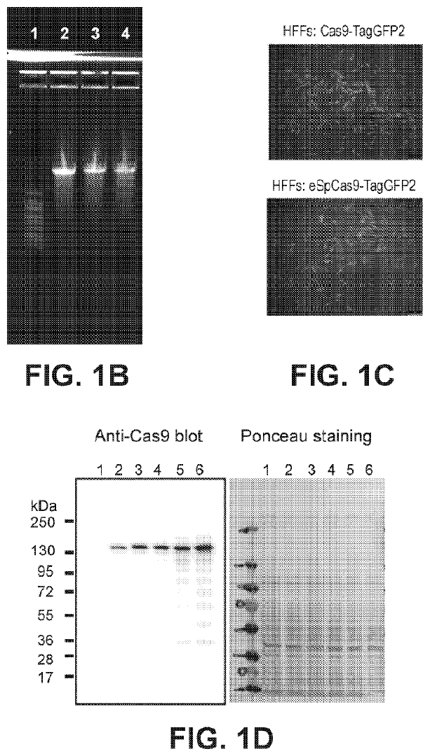

In particular embodiments, the self-replicating RNA vector can be based on a modified Venezuelan equine encephalitis (VEE) virus and can comprise from 5′ to 3′: a 5′ cap, a 5′ UTR, sequence encoding four non-structural replicases from VEE, a promoter, the sequence encoding the CRISPR protein(s), an optional IRES, an optional sequence encoding an E3L protein, an optional IRES, an optional sequence encoding a selectable marker, an alphavirus 3′ UTR, and a poly A tail (see FIG. 1A).

(b) CRISPR Proteins

The self-replicating RNA vector also comprises sequence encoding at least one CRISPR protein. CRISPR proteins, which provide adaptive immunity against invading nucleic acids, are present in various bacteria and archaea. In various embodiments, the CRISPR protein can be a type II Cas9 protein, a type V Cas12 (formerly called Cpf1) protein, a type VI Cas13 (formerly called C2cd) protein, a CasX protein, or a CasY protein.

The CRISPR protein can be from Acaryochloris spp., Acetohalobium spp., Acidaminococcus spp., Acidithiobacillus spp., Acidothermus spp., Akkermansia spp., Alicyclobacillus spp., Allochromatium spp., Ammonifex spp., Anabaena spp., Arthrospira spp., Bacillus spp., Bifidobacterium spp., Burkholderiales spp., Caldicelulosiruptor spp., Campylobacter spp., Candidatus spp., Clostridium spp., Corynebacterium spp., Crocosphaera spp., Cyanothece spp., Deltaproteobacterium spp., Exiguobacterium spp., Finegoldia spp., Francisella spp., Ktedonobacter spp., Lachnospiraceae spp., Lactobacillus spp., Leptotrichia spp., Lyngbya spp., Marinobacter spp., Methanohalobium spp., Microscilla spp., Microcoleus spp., Microcystis spp., Mycoplasma spp., Natranaerobius spp., Neisseria spp., Nitratifractor spp., Nitrosococcus spp., Nocardiopsis spp., Nodularia spp., Nostoc spp., Oenococcus spp., Oscillatoria spp., Parasutterella spp., Pelotomaculum spp., Petrotoga spp., Planctomyces spp., Polaromonas spp., Prevotella spp., Pseudoalteromonas spp., Ralstonia spp., Ruminococcus spp., Staphylococcus spp., Streptococcus spp., Streptomyces spp., Streptosporangium spp., Synechococcus spp., Thermosipho spp., Verrucomicrobia spp., or Wolinella spp. These various CRISPR protein sequences and derivatives thereof are known and can be found in the scientific literature, which is hereby incorporated by reference herein.

In some embodiments, the CRISPR protein can be Streptococcus pyogenes Cas9, Francisella novicida Cas9, Staphylococcus aureus Cas9, Streptococcus thermophilus Cas9, Streptococcus pasteurianus Cas9, Campylobacter jejuni Cas9, Neisseria meningitis Cas9, Neisseria cinerea Cas9, Francisella novicida Cas12, Acidaminococcus sp. Cas12, Lachnospiraceae bacterium ND2006 Cas12a, Leptotrichia wadeii Cas13a, Leptotrichia shahii Cas13a, Prevotella sp. P5-125 Cas13, Ruminococcus flavefaciens Cas13d, Deltaproteobacterium CasX, Planctomyces CasX, or Candidatus CasY. In specific embodiments, the CRISPR protein is Streptococcus pyogenes Cas9 or Staphylococcus aureus Cas9.

The CRISPR protein can be a wild type or naturally-occurring protein. Wild-type CRISPR proteins generally comprise two nuclease domains, e.g., Cas9 proteins comprise RuvC and HNH domains, each of which cleaves one strand of a double-stranded sequence. CRISPR proteins also comprise domains that interact with the guide RNA (e.g., REC1, REC2) or the RNA/DNA heteroduplex (e.g., REC3), and a domain that interacts with the protospacer-adjacent motif (PAM) (i.e., PAM-interacting domain).

Alternatively, the CRISPR protein can be modified or engineered to have altered activity, specificity, and/or stability. For example, the CRISPR protein can be engineered to comprise one or more modifications/mutations (i.e., substitution, deletion, and/or insertion of at least one amino acid). The modified CRISPR protein can have altered catalytic (nuclease) activity, improved target site specificity, decreased off-target effects, altered PAM specificity, increased stability, and the like.

In various embodiments, the CRISPR protein can be a nuclease (i.e., cleave both strands of a double-stranded nucleotide sequence or cleave a single-stranded nucleotide sequence). In other embodiment, CRISPR protein can be a nickase, which cleaves one strand of a double-stranded sequence. The nickase can be engineered via inactivation of one of the nuclease domains of the CRISPR protein. For example, the RuvC domain of a Cas9 protein can be inactivated by mutations such as D10A, D8A, E762A, and/or D986A, or the HNH of a Cas9 protein domain can be inactivated by mutations such as H840A, H559A, N854A, N856A, and/or N863A (with reference to the numbering system of Streptococcus pyogenes Cas9, SpyCas9) to generate a Cas9 nickase (e.g., nCas9). Comparable mutations in other CRISPR proteins can generate nickases (e.g., nCas12). Inactivation of both nuclease domains generates a CRISPR protein with no cleavage activity, i.e., a catalytically inactive or nuclease dead protein (e.g., dCas9, dCas12, and so forth).

The CRISPR protein can also be engineered by one or more amino acid substitutions, deletions, and/or insertions to have improved targeting specificity, improved fidelity, altered PAM specificity, decreased off-target effects, and/or increased stability. Non-limiting examples of one or more mutations that improve targeting specificity, improve fidelity, and/or decrease off-target effects include N497A, R661A, Q695A, K810A, K848A, K855A, Q926A, K1003A, R1060A, and/or D1135E (with reference to the numbering system of SpyCas9).

The RNA vector sequence coding the CRISPR protein can be codon optimized for efficient translation into protein in the eukaryotic cell of interest. Codon optimization programs are available as freeware or from commercial sources. In specific embodiments, the sequence coding the CRISPR protein can be codon optimized for efficient expression in human cells.

Optional Heterologous Domains

In some embodiments, the CRISPR protein can be engineered to comprise at least one heterologous domain, i.e., CRISPR protein can be linked to one or more heterologous domains. The heterologous domain can be a nuclear localization signal (NLS), a cell-penetrating domain, a marker domain (e.g., fluorescent protein), a chromatin modulating motif, an epigenetic modification domain (e.g., a deaminase domain, a histone acetyltransferase domain, and the like), a transcriptional regulation domain, an RNA aptamer binding domain, or a non-CRISPR nuclease domain. In situations in which two or more heterologous domains are fused with a CRISPR protein, the two or more heterologous domains can be the same or they can be different. The one or more heterologous domains can be linked to the CRISPR protein at its N terminal end, the C terminal end, an internal location, or combination thereof. The linkage can be direct via a chemical bond, or the linkage can be indirect via one or more linkers. Suitable linkers are known in the art. In certain embodiments, the linkage can be via a 2A peptide sequence.

In some embodiments the one or more heterologous domains can be a nuclear localization signal (NLS). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO:12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO:16), NQSSNFGPMKGGNFGGRSSGPYGGGGQYFAKPRNQGGY (SEQ ID NO:17), and RMRIZFKNKGKDTAELRRRRVEVSVELRKAKKDEQILKRRNV (SEQ ID NO:18).

In other embodiments, the one or more heterologous domains can be a cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO:24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO:28), KALAWEAKLAKALAKALAKHLAKALAKALKCEA (SEQ ID NO:29), and RQIKIWFQNRRMKWKK (SEQ ID NO:30).

In alternate embodiments, the one or more heterologous domains can be a marker domain. Marker domains include fluorescent proteins and purification or epitope tags. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), orange fluorescent proteins (e.g., mOrange, mKO, Kusabira-Orange, Monomeric Kusabira-Orange, mTangerine, tdTomato), or combinations thereof. The fluorescent protein can comprise tandem repeats of one or more fluorescent proteins (e.g., Suntag). Non-limiting examples of suitable purification or epitope tags include 6× His, FLAG®, HA, GST, Myc, SAM, and the like. Non-limiting examples of heterologous fusions which facilitate detection or enrichment of CRISPR complexes include streptavidin (Kipriyanov et al., Human Antibodies, 1995, 6(3):93-101.), avidin (Airenne et al., Biomolecular Engineering, 1999, 16(1-4):87-92), monomeric forms of avidin (Laitinen et al., Journal of Biological Chemistry, 2003, 278(6):4010-4014), peptide tags which facilitate biotinylation during recombinant production (Cull et al., Methods in Enzymology, 2000, 326:430-440).

In still other embodiments, the one or more heterologous domains can be a chromatin modulating motif (CMM). Non-limiting examples of CMMs include nucleosome interacting peptides derived from high mobility group (HMG) proteins (e.g., HMGB1, HMGB2, HMGB3, HMGN1, HMGN2, HMGN3a, HMGN3b, HMGN4, and HMGN5 proteins), the central globular domain of histone H1 variants (e.g., histone H1.0, H1.1, H1.2, H1.3, H1.4, H1.5, H1.6, H1.7, H1.8, H1.9, and H.1.10), or DNA binding domains of chromatin remodeling complexes (e.g., SWI/SNF (SWItch/Sucrose Non-Fermentable), ISWI (Imitation SWItch), CHD (Chromodomain-Helicase-DNA binding), Mi-2/NuRD (Nucleosome Remodeling and Deacetylase), INO80, SWR1, and RSC complexes, as described in U.S. patent application Ser. No. 16/031,819, the disclosure of which is incorporated by reference herein. Suitable CMMs also can be derived from topoisomerases, helicases, or viral proteins. The source of the CMM can and will vary. CMMs can be from humans, animals (i.e., vertebrates and invertebrates), plants, algae, or yeast.

In yet other embodiments, the one or more heterologous domains can be an epigenetic modification domain. Non-limiting examples of suitable epigenetic modification domains include those with DNA deamination (e.g., cytidine deaminase, adenosine deaminase, guanine deaminase), DNA methyltransferase activity (e.g., cytosine methyltransferase), DNA demethylase activity, DNA amination, DNA oxidation activity, DNA helicase activity, histone acetyltransferase (HAT) activity (e.g., HAT domain derived from E1A binding protein p300), histone deacetylase activity, histone methyltransferase activity, histone demethylase activity, histone kinase activity, histone phosphatase activity, histone ubiquitin ligase activity, histone deubiquitinating activity, histone adenylation activity, histone deadenylation activity, histone SUMOylating activity, histone deSUMOylating activity, histone ribosylation activity, histone deribosylation activity, histone myristoylation activity, histone demyristoylation activity, histone citrullination activity, histone alkylation activity, histone dealkylation activity, or histone oxidation activity. In specific embodiments, the epigenetic modification domain can comprise cytidine deaminase activity, adenosine deaminase activity, histone acetyltransferase activity, or DNA methyltransferase activity.

In other embodiments, the one or more heterologous domains can be a transcriptional regulation domain (i.e., a transcriptional activation domain or transcriptional repressor domain). Suitable transcriptional activation domains include, without limit, herpes simplex virus VP16 domain, VP64 (i.e., four tandem copies of VP16), VP160 (i.e., ten tandem copies of VP16), NFκB p65 activation domain (p65) , Epstein-Barr virus R transactivator (Rta) domain, VPR VP64+p65+Rta), p300-dependent transcriptional activation domains, p53 activation domains 1 and 2, heat-shock factor 1 (HSF1) activation domains, Smad4 activation domains (SAD), cAMP response element binding protein (CREB) activation domains, E2A activation domains, nuclear factor of activated T-cells (NFAT) activation domains, or combinations thereof. Non-limiting examples of suitable transcriptional repressor domains include Kruppel-associated box (KRAB) repressor domains, Mxi repressor domains, inducible cAMP early repressor (ICER) domains, YY1 glycine rich repressor domains, Sp1-like repressors, E(spI) repressors, IκB repressors, Sin3 repressors, methyl-CpG binding protein 2 (MeCP2) repressors, or combinations thereof. Transcriptional activation or transcriptional repressor domains can be genetically fused to the Cas9 protein or bound via noncovalent protein-protein, protein-RNA, or protein-DNA interactions.

In further embodiments, the one or more heterologous domains can be an RNA aptamer binding domain (Konermann et al., Nature, 2015, 517(7536):583-588; Zalatan et al., Cell, 2015, 160(1-2):339-50). Examples of suitable RNA aptamer protein domains include MS2 coat protein (MCP), PP7 bacteriophage coat protein (PCP), Mu bacteriophage Com protein, lambda bacteriophage N22 protein, stem-loop binding protein (SLBP), Fragile X mental retardation syndrome-related protein 1 (FXR1), proteins derived from bacteriophage such as AP205, BZ13, f1, f2, fd, fr, ID2, JP34/GA, JP501, JP34, JP500, KU1, M11, M12, MX1, NL95, PP7, ϕCb5, ϕCb8r, ϕCb12r, ϕCb23r, Qβ, R17, SP-β, TW18, TW19, and VK, fragments thereof, or derivatives thereof.

In yet other embodiments, the one or more heterologous domains can be a non-CRISPR nuclease domain. Suitable nuclease domains can be obtained from any endonuclease or exonuclease. Non-limiting examples of endonucleases from which a nuclease domain can be derived include, but are not limited to, restriction endonucleases and homing endonucleases. In some embodiments, the nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In some embodiments, the nuclease domain can be a FokI nuclease domain or a derivative thereof. The type II-S nuclease domain can be modified to facilitate dimerization of two different nuclease domains. For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. In specific embodiments, the FokI nuclease domain can comprise a first FokI half-domain comprising Q486E, I499L, and/or N496D mutations, and a second FokI half-domain comprising E490K, I538K, and/or H537R mutations.

### (II) Complexes Comprising Self-Replicating RNA Vector and Guide RNA

Another aspect of the present disclosure encompasses complexes comprising any of the self-replicating RNA vectors described above in section (I) and at least one guide RNA, wherein the guide RNA is designed to complex with the CRISPR protein coded by the self-replicating RNA vector.

A guide RNA interacts with the CRISPR protein and a target sequence in the nucleic acid of interest and guides the CRISPR protein to the target sequence. The target sequence has no sequence limitation except that the sequence is adjacent to a protospacer adjacent motif (PAM) sequence. CRISPR proteins can recognize different PAM sequences. For example, PAM sequences for Cas9 proteins include 5′-NGG, 5′-NGGNG, 5′-NNAGAAW, 5′-NNNNGATT, and 5-NNNNRYAC, and PAM sequences for Cas12 proteins include 5′-TTN and 5′-TTTV, wherein N is defined as any nucleotide, R is defined as either G or A, W is defined as either A or T, Y is defined an either C or T, and V is defined as A, C, or G. In general, Cas9 PAMs are located 3′ of the target sequence, and Cas12 PAMs are located 5′ of the target sequence.

Guide RNA are engineered to complex with a specific CRISPR protein. In general, a guide RNA comprises (i) a CRISPR RNA (crRNA) that contains a guide sequence at the 5′ end that hybridizes with the target sequence, and (ii) a transacting crRNA (tracrRNA) sequence that interacts with the CRISPR protein. The crRNA guide sequence of each guide RNA is different (i.e., is sequence specific). The tracrRNA sequence is generally the same in guide RNAs designed to complex with a specific CRISPR protein.

The crRNA guide sequence is designed to hybridize with a target sequence (i.e., protospacer) in a sequence of interest. In general, the complementarity between the crRNA and the target sequence is at least 80%, at least 85%, at least 90%, at least 95%, or at least 99%. In specific embodiments, the complementarity is complete (i.e., 100%). In various embodiments, the length of the crRNA guide sequence can range from about 15 nucleotides to about 25 nucleotides. For example, the crRNA guide sequence can be about 15, 16, 17, 18, 19, 20, 21, 22, 23, 24, or 25 nucleotides in length. In specific embodiments, the crRNA is about 19, 20, or 21 nucleotides in length. In one embodiment, the crRNA guide sequence has a length of 20 nucleotides.

The guide RNA comprises repeat sequence that forms at least one stem loop structure, which interacts with the CRISPR protein, and 3′ sequence that generally remains single-stranded. The length of each loop and stem can vary. For example, the loop can range from about 3 to about 10 nucleotides in length, and the stem can range from about 6 to about 20 base pairs in length. The stem can comprise one or more bulges of 1 to about 10 nucleotides. The length of the single-stranded 3′ region can vary. The tracrRNA sequence in the guide RNA generally is based upon the sequence of wild type tracrRNA that interact with the wild-type CRISPR protein. The wild-type sequence can be modified to facilitate secondary structure formation, increased secondary structure stability, facilitate expression in eukaryotic cells, and so forth. For example, one or more nucleotide changes can be introduced into the guide RNA coding sequence. The tracrRNA sequence can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides.

In some embodiments, the guide RNA can be a single molecule (e.g., a single guide RNA (sgRNA) or 1-piece sgRNA), wherein the crRNA sequence is linked to the tracrRNA sequence. In some embodiments, the guide RNA can be two separate molecules (e.g., 2-piece gRNA). A first molecule comprising the crRNA that contains 3′ sequence (comprising from about 6 to about 20 nucleotides) that is capable of base pairing with the 5′ end of a second molecule, wherein the second molecule comprises the tracrRNA that contains 5′ sequence (comprising from about 6 to about 20 nucleotides) that is capable of base pairing with the 3′ end of the first molecule.

In some embodiments, the tracrRNA sequence of the guide RNA can be modified to comprise one or more aptamer sequences (Konermann et al., Nature, 2015, 517(7536):583-588; Zalatan et al., Cell, 2015, 160(1-2):339-50). Suitable aptamer sequences include those that bind adaptor proteins chosen from MCP, PCP, Com, SLBP, FXR1, AP205, BZ13, f1, f2, fd, fr, ID2, JP34/GA, JP501, JP34, JP500, KU1, M11, M12, MX1, NL95, PP7, ϕCb5, ϕCb8r, ϕCb12r, ϕCb23r, Qβ, R17, SP-β, TW18, TW19, VK, fragments thereof, or derivatives thereof. Those of skill in the art appreciate that the length of the aptamer sequence can vary.

In other embodiments, the guide RNA can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

The guide RNA can comprise standard ribonucleotides and/or modified ribonucleotides. In some embodiment, the guide RNA can comprise standard or modified deoxyribonucleotides. In embodiments in which the guide RNA is enzymatically synthesized (i.e., in vivo or in vitro), the guide RNA generally comprises standard ribonucleotides. In embodiments in which the guide RNA is chemically synthesized, the guide RNA can comprise standard or modified ribonucleotides and/or deoxyribonucleotides. Modified ribonucleotides and/or deoxyribonucleotides include base modifications (e.g., pseudouridine, 2-thiouridine, N6-methyladenosine, and the like) and/or sugar modifications (e.g., 2′-O-methy, 2′-fluoro, 2′-amino, locked nucleic acid (LNA), and so forth). The backbone of the guide RNA can also be modified to comprise phosphorothioate linkages, boranophosphate linkages, or peptide nucleic acids.

### (III) Eukaryotic Cells

Another aspect of the present disclosure comprises eukaryotic cells or cell lines comprising any one of the self-replicating RNA vectors described above in section (I). That is, the eukaryotic cells or cell lines have been transfected with one of the self-replicating RNA vectors. In some embodiments, the eukaryotic cells or cell lines can further comprise at least one guide RNA that is engineered to complex with the CRISPR protein coded by the RNA vector.

The eukaryotic cell or cell line can be a human cell, a non-human mammalian cell, a non-mammalian vertebrate cell, an invertebrate cell, a plant cell, or a single cell eukaryotic organism. Examples of suitable eukaryotic cells are detailed below in section (V)(c). The eukaryotic cell can be in vitro, ex vivo, or in vivo.

### (IV) Plasmid Vectors Encoding the Self-Replicating RNA

A further aspect of the present disclosure provides plasmid vectors encoding the self-replicating RNA described above in section (I). In particular, the plasmid vector comprises sequence encoding the non-structural replication complex proteins from an alphavirus, sequence encoding the CRISPR protein, as well as additional viral sequences such as 5′ UTR, subgenomic promoter, and 3′ UTR, optional selectable marker sequence, optional interferon inhibitor sequence, optional IRES, etc.

In general, the plasmid vectors encoding the self-replicating RNA are DNA vectors. The sequence encoding the self-replicating RNA can be operably linked to a promoter sequence that is recognized by a phage RNA polymerase for in vitro RNA synthesis. For example, the promoter sequence can be a T7, T3, or SP6 promoter sequence or a variation of a T7, T3, or SP6 promoter sequence. The promoter sequence can be wild type or it can be modified for more efficient or efficacious expression. The plasmid vector can further comprise at least one transcriptional termination sequence, as well as at least one origin of replication and/or selectable marker sequence (e.g., antibiotic resistance genes) for propagation in bacterial cells. The plasmid vector can be derived from pUC, pBR322, pET, pBluescript, or variants thereof. Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

Upon in vitro synthesis of the self-replicating RNA, the RNA can be purified, 5′ capped, and polyadenylated using standard procedures or commercially available kits.

### (V) Methods for Genome Editing

A further aspect of the present disclosure encompasses methods for genome editing in eukaryotic cells. In general, the methods comprise introducing into the eukaryotic cell of interest any one of the self-replicating RNA vectors described above in section (I) and at least one guide RNA that is engineered to complex with the CRISPR protein coded by the RNA vector. The method can also comprise introducing into the eukaryotic a complex as specified above in section (II). A CRISPR system comprises a CRISPR protein and guide RNA.

In embodiments in which the CRISPR protein comprises nuclease or nickase activity, the genome editing can comprise a substitution of at least one nucleotide, a deletion of at least one nucleotide, and/or an insertion of at least one nucleotide. In some iterations, the method comprises introducing into the eukaryotic cell one CRISPR system comprising nuclease activity or two CRISPR systems comprising nickase activity and no donor polynucleotide, such that the CRISPR system or systems introduce a double-stranded break in the target site in the sequence of interest and repair of the double-stranded break by cellular DNA repair processes introduces at least one nucleotide change (i.e., indel), thereby inactivating the sequence (i.e., gene knock-out). In other iterations, the method comprises introducing into the eukaryotic cell a CRISPR system comprising nuclease activity or two CRISPR systems comprising nickase activity, as well as the donor polynucleotide, such that the CRISPR system or systems introduce a double-stranded break in the target site in the sequence of interest and repair of the double-stranded break by cellular DNA repair processes leads to insertion or exchange of sequence in the donor polynucleotide into the target site in the sequence of interest (i.e., gene correction or gene knock-in).

In embodiments, in which the CRISPR protein comprises epigenetic modification activity or transcriptional regulation activity, the genome editing can comprise a conversion of at least one nucleotide in or near the target site (i.e., base editing), a modification of at least one nucleotide in or near the target site, a modification of at least one histone protein in or near the target site, and/or a change in transcription in or near the target site in the chromosomal sequence.

(a) Introduction into the Cell

As mentioned above, the method comprises introducing into the eukaryotic cell at least one self-replicating RNA vector described above in section (I), at least one guide RNA that is engineered to complex with the CRISPR protein coded by the RNA vector, and an optional donor polynucleotide. The molecules can be introduced into the cell of interest by a variety of means.

For example, self-replicating RNA vector, guide RNA, and optional donor polynucleotide can be transfected into the cell of interest. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001). In other embodiments, the molecules can be introduced into the cell by microinjection. For example, the molecules can be injected into the cytoplasm or nuclei of the cells of interest. The amount of each molecule introduced into the cell can vary, but those skilled in the art are familiar with means for determining the appropriate amount.

The various molecules can be introduced into the cell simultaneously or sequentially. For example, the self-replicating RNA vector and the guide RNA can be introduced at the same time. Alternatively, one can be introduced first and then the other can be introduced later into the cell.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Umov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

(b) Optional Donor Polynucleotide

In embodiments in which the CRISPR protein comprises nuclease or nickase activity, the method can further comprise introducing at least one donor polynucleotide into the cell. The donor polynucleotide can be single-stranded or double-stranded, linear or circular, and/or RNA or DNA. In some embodiments, the donor polynucleotide can be a vector, e.g., a plasmid vector.

The donor polynucleotide comprises at least one donor sequence. In some aspects, the donor sequence of the donor polynucleotide can be a modified version of an endogenous or native chromosomal sequence. For example, the donor sequence can be essentially identical to a portion of the chromosomal sequence at or near the sequence targeted by the CRISPR system, but which comprises at least one nucleotide change. Thus, upon integration or exchange with the native sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the “gene correction” integration of the modified sequence, the cell can produce a modified gene product from the targeted chromosomal sequence.

In other aspects, the donor sequence of the donor polynucleotide can be an exogenous sequence. As used herein, an “exogenous” sequence refers to a sequence that is not native to the cell, or a sequence whose native location is in a different location in the genome of the cell. For example, the exogenous sequence can comprise protein coding sequence, which can be operably linked to an exogenous promoter control sequence such that, upon integration into the genome, the cell is able to express the protein coded by the integrated sequence. Alternatively, the exogenous sequence can be integrated into the chromosomal sequence such that its expression is regulated by an endogenous promoter control sequence. In other iterations, the exogenous sequence can be a transcriptional control sequence, another expression control sequence, an RNA coding sequence, and so forth. As noted above, integration of an exogenous sequence into a chromosomal sequence is termed a “knock in.”

As can be appreciated by those skilled in the art, the length of the donor sequence can and will vary. For example, the donor sequence can vary in length from several nucleotides to hundreds of nucleotides to hundreds of thousands of nucleotides.

Typically, the donor sequence in the donor polynucleotide is flanked by an upstream sequence and a downstream sequence, which have substantial sequence identity to sequences located upstream and downstream, respectively, of the sequence targeted by the CRISPR system. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the sequence targeted by the CRISPR system. Similarly, the downstream sequence refers to a nucleic acid sequence that shares substantial sequence identity with a chromosomal sequence downstream of the sequence targeted by the CRISPR system. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the target sequence. In an exemplary embodiment, the upstream and downstream sequences in the donor polynucleotide can have about 95% or 100% sequence identity with chromosomal sequences upstream or downstream to the sequence targeted by the CRISPR system.

In some embodiments, the upstream sequence shares substantial sequence identity with a chromosomal sequence located immediately upstream of the sequence targeted by the CRISPR system. In other embodiments, the upstream sequence shares substantial sequence identity with a chromosomal sequence that is located within about one hundred (100) nucleotides upstream from the target sequence. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the target sequence. In some embodiments, the downstream sequence shares substantial sequence identity with a chromosomal sequence located immediately downstream of the sequence targeted by the CRISPR system. In other embodiments, the downstream sequence shares substantial sequence identity with a chromosomal sequence that is located within about one hundred (100) nucleotides downstream from the target sequence. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the target sequence.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In specific embodiments, upstream and downstream sequences can range in length from about 50 to about 1500 nucleotides.

(c) Cell Types

A variety of eukaryotic cells are suitable for use in the methods disclosed herein. For example, the cell can be a human cell, a non-human mammalian cell, a non-mammalian vertebrate cell, an invertebrate cell, an insect cell, a plant cell, a yeast cell, or a single cell eukaryotic organism. In some embodiments, the cell can be a primary cell that was isolated directly from a specific tissue. In other embodiments, the cell can be a cell line cell. In still other embodiments, the cell can be a one cell embryo. For example, a non-human mammalian embryo including rat, hamster, rodent, rabbit, feline, canine, ovine, porcine, bovine, equine, and primate embryos. In still other embodiments, the cell can be a stem cell such as embryonic stem cells, ES-like stem cells, fetal stem cells, adult stem cells, induced pluripotent stem cell, and the like. In one embodiment, the stem cell is not a human embryonic stem cell. Furthermore, the stem cells may include those made by the techniques disclosed in WO2003/046141, which is incorporated herein in its entirety, or Chung et al. (Cell Stem Cell, 2008, 2:113-117). In various embodiments, the cell can be in vitro (i.e., in culture), ex vivo (i.e., within tissue isolated from an organism), or in vivo (i.e., within an organism). In exemplary embodiments, the cell is a mammalian cell or mammalian cell line. In particular embodiments, the cell is a human cell or human cell line.

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells, baby hamster kidney (BHK) cells; mouse myeloma NS0 cells, human primary fibroblasts, human foreskin fibroblasts, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells. An extensive list of mammalian cell lines may be found in the American Type Culture Collection catalog (ATCC, Manassas, Va.).

### (VI) Applications

The compositions and methods disclosed herein can be used in a variety of therapeutic, diagnostic, industrial, and research applications. In some embodiments, the present disclosure can be used to modify any chromosomal sequence of interest in a cell, animal, or plant in order to model and/or study the function of genes, study genetic or epigenetic conditions of interest, or study biochemical pathways involved in various diseases or disorders. For example, transgenic organisms can be created that model diseases or disorders, wherein the expression of one or more nucleic acid sequences associated with a disease or disorder is altered. The disease model can be used to study the effects of mutations on the organism, study the development and/or progression of the disease, study the effect of a pharmaceutically active compound on the disease, and/or assess the efficacy of a potential gene therapy strategy.

In other embodiments, the compositions and methods can be used to perform efficient and cost effective functional genomic screens, which can be used to study the function of genes involved in a particular biological process and how any alteration in gene expression can affect the biological process, or to perform saturating or deep scanning mutagenesis of genomic loci in conjunction with a cellular phenotype. Saturating or deep scanning mutagenesis can be used to determine critical minimal features and discrete vulnerabilities of functional elements required for gene expression, drug resistance, and reversal of disease, for example.

In further embodiments, the compositions and methods disclosed herein can be used for diagnostic tests to establish the presence of a disease or disorder and/or for use in determining treatment options. Examples of suitable diagnostic tests include detection of specific mutations in cancer cells (e.g., specific mutation in EGFR, HER2, and the like), detection of specific mutations associated with particular diseases (e.g., trinucleotide repeats, mutations in β-globin associated with sickle cell disease, specific SNPs, etc.), detection of hepatitis, detection of viruses (e.g., Zika), and so forth.

In additional embodiments, the compositions and methods disclosed herein can be used to correct genetic mutations associated with a particular disease or disorder such as, e.g., correct globin gene mutations associated with sickle cell disease or thalassemia, correct mutations in the adenosine deaminase gene associated with severe combined immune deficiency (SCID), reduce the expression of HTT, the disease-causing gene of Huntington's disease, or correct mutations in the rhodopsin gene for the treatment of retinitis pigmentosa. Such modifications may be made in cells ex vivo.

In still other embodiments, the compositions and methods disclosed herein can be used to generate crop plants with improved traits or increased resistance to environmental stresses. The present disclosure can also be used to generate farm animal with improved traits or production animals. For example, pigs have many features that make them attractive as biomedical models, especially in regenerative medicine or xenotransplantation.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

As used herein, the term “CRISPR/Cas system” or “Cas9 system” refers to a complex comprising a Cas9 protein (i.e., nuclease, nickase, or catalytically dead protein) and a guide RNA.

The term “endogenous sequence,” as used herein, refers to a chromosomal sequence that is native to the cell.

As used herein, the term “exogenous” refers to a sequence that is not native to the cell, or a chromosomal sequence whose native location in the genome of the cell is in a different chromosomal location.

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The term “nickase” refers to an enzyme that cleaves one strand of a double-stranded nucleic acid sequence (i.e., nicks a double-stranded sequence). For example, a nuclease with double strand cleavage activity can be modified by mutation and/or deletion to function as a nickase and cleave only one strand of a double-stranded sequence.

The term “nuclease,” as used herein, refers to an enzyme that cleaves both strands of a double-stranded nucleic acid sequence or cleaves a single-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The terms “target sequence,” and “target site” are used interchangeably to refer to the specific sequence in the nucleic acid of interest (e.g., chromosomal DNA or cellular RNA) to which the CRISPR system is targeted, and the site at which the CRISPR system modifies the nucleic acid or protein(s) associated with the nucleic acid.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1. Construction of Self-Replicating Cas9 RNA Vectors

A synthetic, polycistronic, self-replicating RNA encoding Cas9 was generated based on a modified Venezuelan equine encephalitis (VEE) virus in which the structural genes have been removed (i.e., Simplicon™ Cloning Vector E3L; MilliporeSigma). The cDNAs of Cas9-T2A-TagGFP2, eSpCas9-T2A-GFP2, and eSpCas9 were amplified with PCR using pVAV-Cas9-2A-GFP plasmid (encodes wild type SpCas9), CMV-eSpCas9-2A-GFP plasmid, and SpCas9-Blasticidin Lenti plasmid as templates, respectively. eSpCas9 is an engineered version of wild type SpCas9 modified to enhance on-target fidelity without loss of cleavage efficiency (Slaymaker et al., Science. 2016, 351(6268):84-8). Then each cDNA was cloned into NdeI/NotI sites of the Simplicon™ Vector and were named T7-VEE-Cas9-TagGFP2 (SEQ ID NO:31), T7-VEE-eSpCas9-TagGFP2 (SEQ ID NO:32), and T7-VEE-eSpCas9 (SEQ ID NO:33), respectively. The scheme of each VEE-Cas9 RNA is shown in FIG. 1A.

For RNA synthesis, each VEE-Cas9 plasmid and B18R-E3L plasmid were linearized with MluI and BamHI digestion, respectively, to generate templates for RNA synthesis. RNA synthesis and 5′-capping of were performed using the RiboMAX Large Scale RNA Production System-T7 (Promega) kit in the presence of CleanCap® Reagent AG (Trilink) for 2 hr at 37° C. For B18R-E3L RNA synthesis, an additional ˜150 bases of poly(A) tail was added by poly(A) polymerase (CELLSCRIPT) for 30 min at 37° C. Following purification and precipitation with the 2.5 M ammonium acetate, RNAs were resuspended in the RNA Storage Solution (Ambion) at 1 μg/μl concentration and stored at −80° C. The VEE-Cas9 RNAs were analyzed by agarose gel electrophoresis (FIG. 1B). The VEE-Cas9 RNA bands showed the strongest intensity at the predicted size (˜15 kb) with minimum degraded bands.

### Example 2. Transfection of Self-Replicating Cas9 RNAs

These VEE-Cas9 RNAs were co-transfected into cells along with B18R-E3L RNA , which inhibits the interferon (IFN) responses caused by RNA transfection and replication.

Human foreskin fibroblasts (HFFs) and HEK293T cells were cultured in DMEM containing 10% FBS, MEM Non-Essential Amino Acids (NEAA), pyruvate, penicillin, and streptomycin. Cells were passaged one day before the transfection so they were at 30-60% confluency on the day of transfection. Each VEE-Cas9 RNA was co-transfected with B18R-E3L RNA at 1:1 ratio (1 microgram each for 1 well of 6-well plate) into cells with Lipofectamine MessengerMax transfection reagent (Thermofisher).

As shown in FIG. 1C, TagGFP2 (GFP) expression was observed in Cas9-TagGFP2 and eSpCas9-TagGFP2 transfected cells, and expression of each Cas9 protein was confirmed by Western blotting (FIG. 1D). These data indicate that the self-replication RNA technology allows for expression of Cas9 in human cells.

### Example 3. Non-Integrative Cas9 Genome Editing

Next, targeted genome editing was examined using the VEE-Cas9 RNAs in combination with chemically synthesized gRNAs. Commercially available 2-piece synthetic RNAs (crRNA:tracrRNA complex) and 1-piece synthetic single gRNA (sgRNA) targeting K-Ras and EMX-1 were purchased from Thermofisher Scientific. The crRNA sequences including PAM sequence (underlined) for K-Ras and EMX-1 targets are 5′-TAGTTGGAGCTGGTGGCGTAGG (SEQ ID NO:34) and 5′-GAGTCCGAGCAGAAGAAGAAGGG (SEQ ID NO:35), respectively.

Initially, HFF cells were co-transfected with VEE-Cas9-TagGFP2, B18R-E3L RNA (as described above), along with either 1-piece gRNA or 2-piece gRNA. Cells were collected 2-6 days after transfection and indels were detected with Guide-iT Mutation Detection kit (Clontech). The efficiency of DNA cleavage was calculated with ChemiDoc™ Imaging System. The target region for K-Ras (340 bp) and EMX-1 (410 bp) genes were amplified with PCR using primer sets of 5′-GATACACGTCTGCAGTCAACTG (SEQ ID NO:36)/5′-GCATATTACTGGTGCAGGACC (SEQ ID NO:37) and 5′-GCCTGAGTGTTGAGGCCCCA (SEQ ID NO:38)/5′-GTCCCTCTGTCAATGGCGGC (SEQ ID NO:39), respectively.

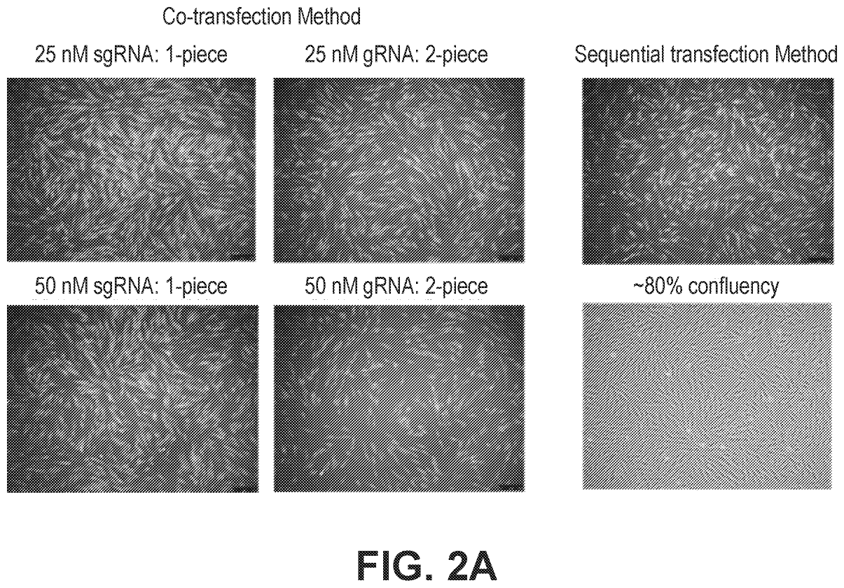

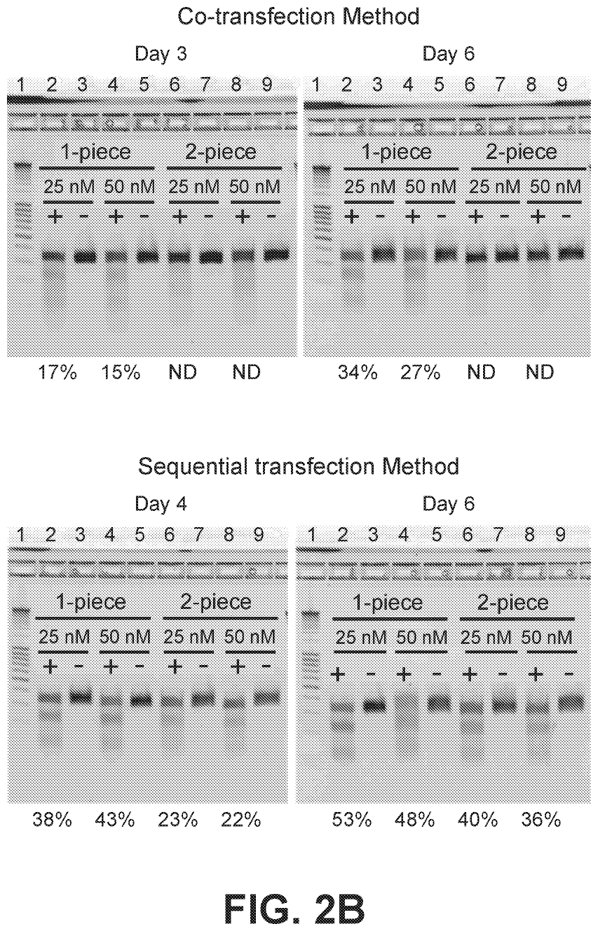

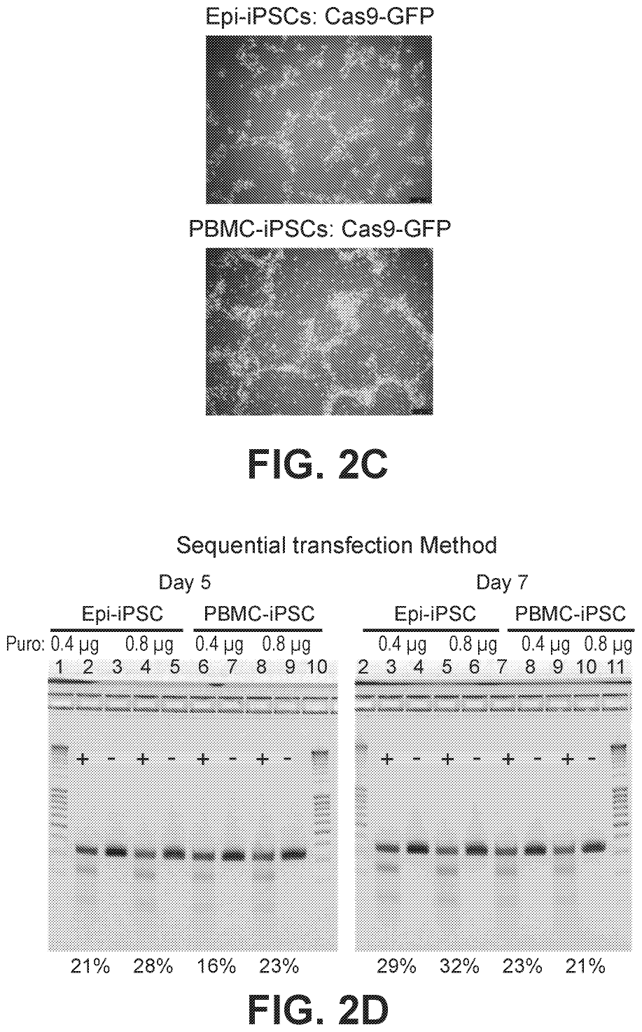

As shown in FIG. 2A, GFP expression was observed using either 1-piece or 2-piece gRNAs, although GFP expression was reduced in the 2-piece gRNA transfected cells. More than 15% cleavage was observed in 1-piece sgRNA transfected cells, whereas cleavage was not detected in 2-piece gRNA transfected cells on day 3 (two days after the transfection) (FIG. 2B). Puromycin selection was performed to remove the Cas9 negative cells, and the efficiency of cleavage was increased in 1-piece sgRNA transfected cells, but cleavage was still not detected in 2-piece gRNA transfected cells (FIG. 2B, day 6).

To increase the efficiency of editing, cells were sequentially transfected with VEE-Cas9 RNA and gRNA. For this experiment, VEE-Cas9 and B18R-E3L RNA were co-transfected on day 1, and then gRNA was transfected on day 2 with Lipofectamine RNAiMax transfection reagent (Thermofisher). As shown in FIG. 2B, higher efficiency of genome editing was obtained with sequential transfection using either the 1-piece or 2-piece gRNA on day 4 (two days after gRNA transfection), and a further increases of efficiency were obtained after puromycin selection (day 6). Two different amounts of gRNA (25 nM and 50 nM) were tested, but no significant difference was observed (FIG. 2B).

Next, VEE-Cas9 genome editing was tested in human iPSC cell lines by the sequential transfection method using the 1-piece sgRNA. Epitherial-1 iPSC or PBMC-iPSC (CD34+ cord blood iPSC) were cultured on laminin coated wells in the presence of mTeSRTM-1 culture medium (Stemcell Technologies). The iPSC cells were transfected as described above. GFP expression was obtained in both human iPSC cell lines (FIG. 2C), and the efficiency of genome editing ranged from 16-32% in both cell lines (FIG. 2D).

### Example 4. Comparison of Self-Replicating Cas9 RNA with Other Expression Systems

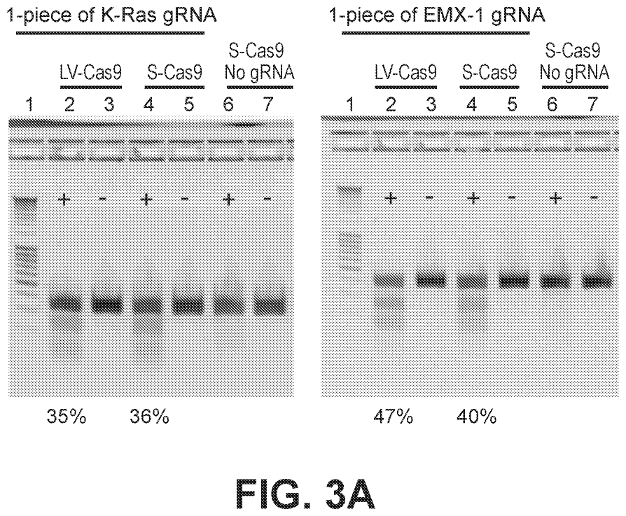

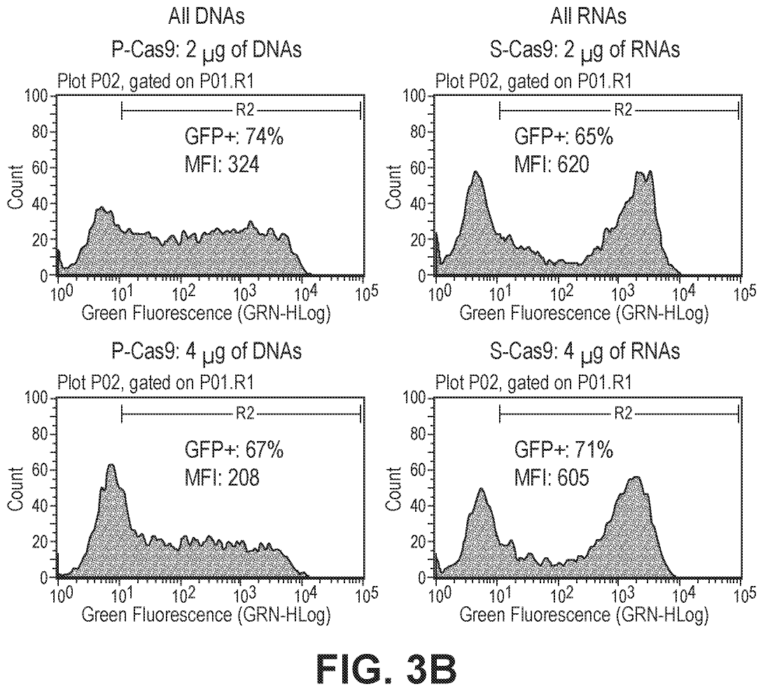

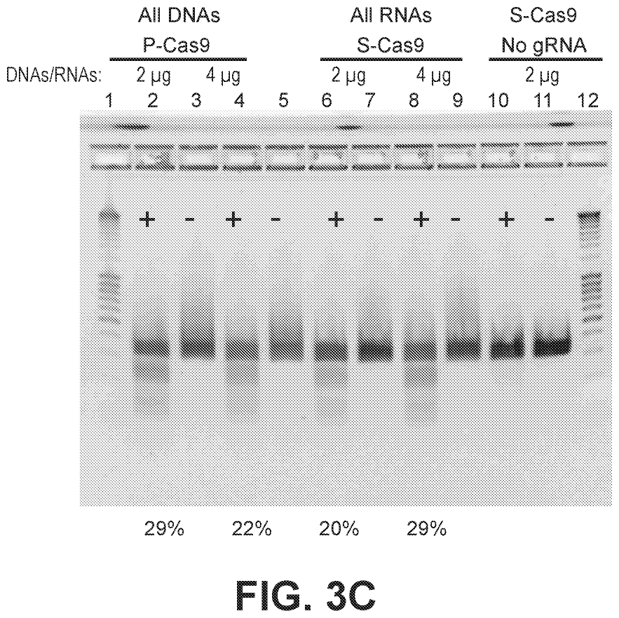

Efficiency of genome editing was compared between VEE-Cas9 RNA and lentivirus Cas9. The lentivirus vector has a blasticidin selection marker. Thus, lentivirus Cas9 (LV-Cas9) (at MOI=3) or VEE-Cas9-TagGFP2 (S-Cas9) were introduced into HFFs, and then the cells were selected with blasticidin (4 μg/mL) or puromycin (0.8 μg/mL), respectively, for a week. After selection, both cells were passaged for the transfection of 1-piece sgRNAs on next day. Similar efficiency of genome editing was observed with both of the Cas9 expression methods at the K-Ras target (35% and 36% respectively) and the EMX-1 target (47% and 40%, respectively) (FIG. 3A).

Next, VEE-Cas9 RNA was compared with plasmid Cas9 encoding Cas9-TagGFP2. HEK293T cells were co-transfected with plasmid Cas9 and plasmid encoding K-Ras-gRNA (all DNA components) or HEK293T cells were co-transfected with VEE-Cas9-TagGFP2, B18R-E3L RNA, and 1-piece K-Ras sgRNA (all RNA components). As shown in FIG. 3B, transfection of all DNA components or all RNA components resulted in similar percentages of GFP positive cells (65-74%). The efficiency of genome editing was also similar (20-29%) with the two methods (FIG. 3C). These data indicate that genome editing with VEE-Cas9 RNA is comparable to that provided by lentivirus and plasmid Cas9 expression tools.

### Example 5. Long Term Expression of Self-Replicating Cass9 RNA

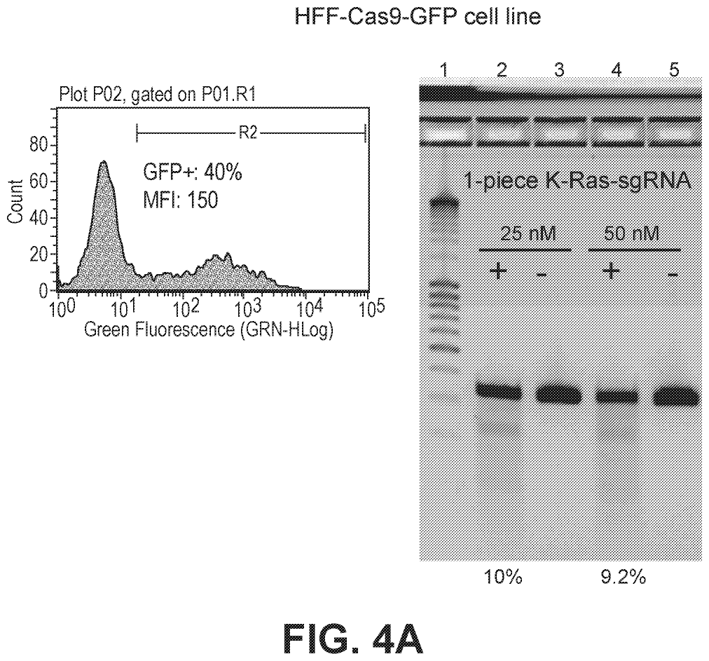

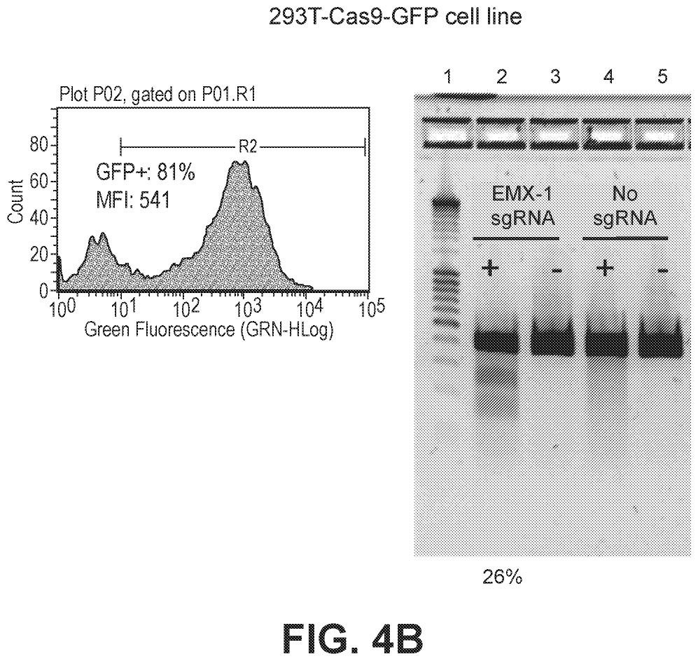

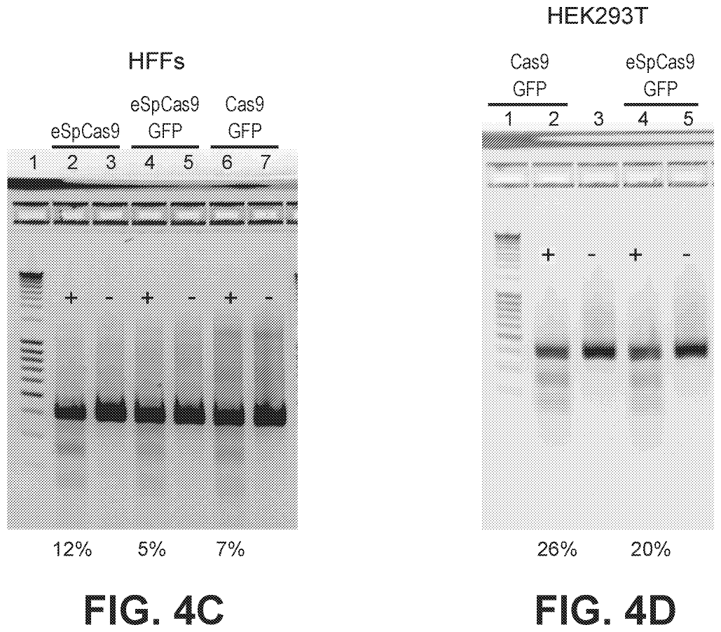

VEE-Cas9-TagGFP2 RNA and B18R-E3L RNA were co-transfected into either HFFs or HEK293T cells and the cells were maintained under puromycin selection in the presence of B18R protein. After a month and four cell passages, the HFF cells were co-transfected with 1-piece K-Ras sgRNA. As shown in FIG. 4A, about 40% of the HFF cells were GFP positive and a genome editing efficiency of about 10% was obtained. After a month and eight cell passages, the HEK293T were co-transfected with 1-piece EMX-1 sgRNA. It was found that about 81% of the HEK293T cells were GFP positive and the genome editing efficiency was about 26% (FIG. 4B). These data suggest that VEE-Cas9 RNA allows for the generation of Cas9 expressing cell lines without manipulating host cell genome, and said cell lines are available for the targeted genome editing.

The efficiency of genome editing was compared among the three VEE-Cas9 RNA vectors prepared in Example 1. As shown in FIGS. 4C and 4D, similar efficiency of genome editing was obtained with eSpCas9, eSpCas9-TagGFP2, and Cas9-TagGFP2 in HFFs and HEK293T cells (e.g., 5-12% in HFFs, 20-26% in HEK293T cells).

### Example 6. Genome Editing with Self-Replicating D10A-Cas9 RNA

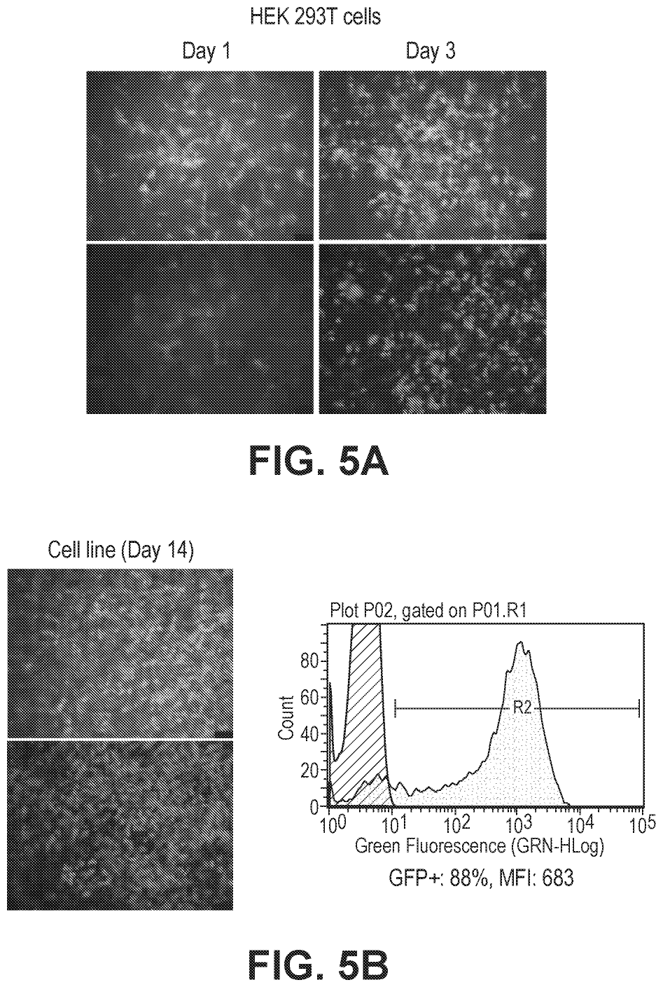

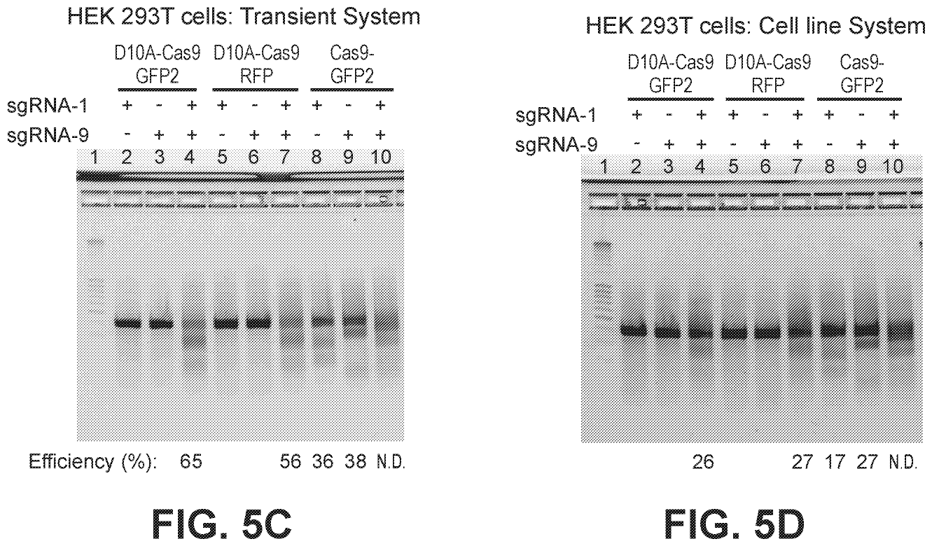

D10A mutation on Cas9 protein results in single-strand cleavage instead of double-strand cleavage. Therefore, it considered performing genome editing with less off-target cleavage and useful for precise genome editing. To examine the availability of D10A-Cas9 with a self-replicative RNA, we generated a new construct of Cas9 with a D10A point mutation. FIG. 5A shows the expression of D10A-Cas9-TagGFP2 and D10A-Cas9-TagRFP in 293T cells on day 1 and 3, and Cas9 expressing cell lines (293T) were generated (FIG. 5B). For testing the availability of D10A-Cas9, two kinds of sgRNAs (sgRNA-1 and 9) at the EMX1 gene locus were transfected to generate the double-strand break. As shown in FIG. 5C, the genome editing was observed when two kinds of sgRNAs were transfected into Cas9-D10A mutant expressing cells, while no genome editing was observed with one sgRNA was transfected. The same results were observed in D10A-Cas9 cell lines (FIG. 5D). These data show that self-replicating D10A-Cas9 is available for precise genome editing.

### Example 7. Insertion of DNA Oligo and GFP DNA Fragment with Self-Replicative Cas9 RNA

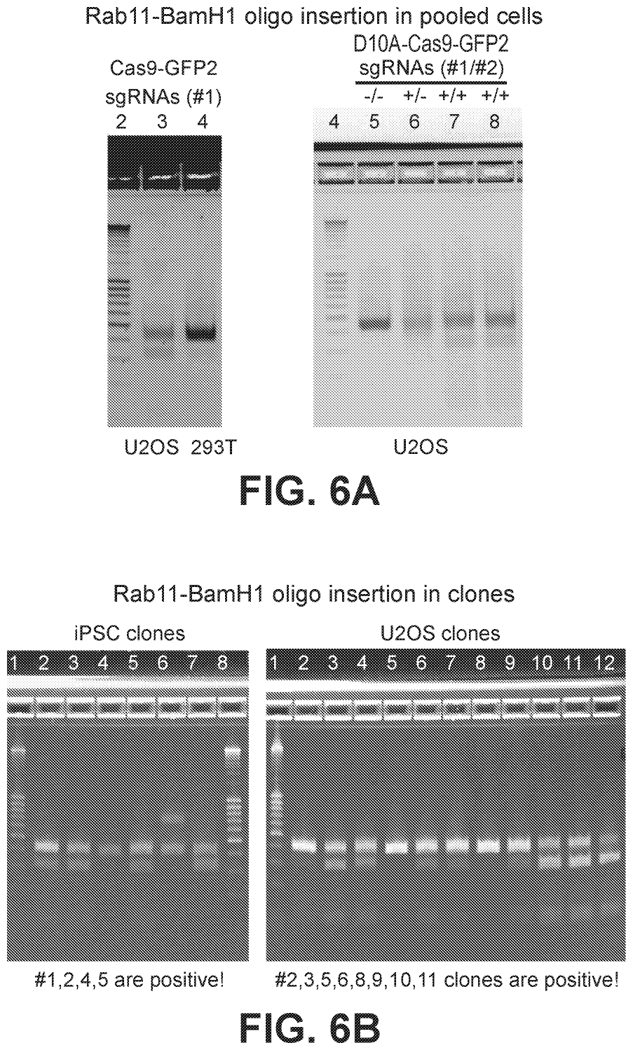

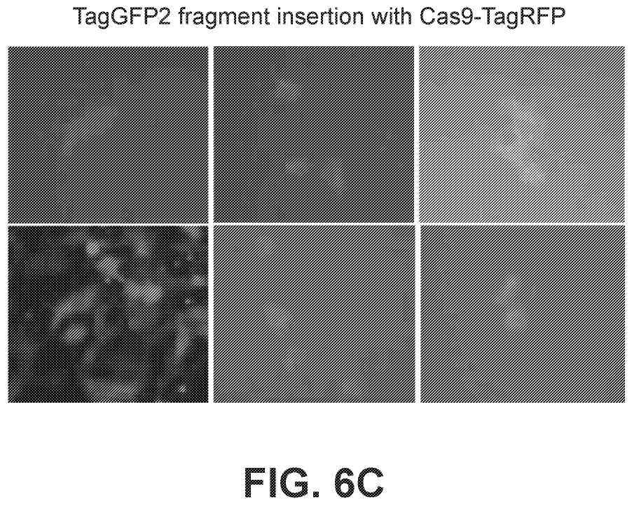

Next, we tested for DNA insertion at the Cas9 cleavage site. First, we inserted DNA oligo having a BamHI restriction enzyme site (BamHl oligo) into the Rab11 gene locus. A self-replicative Cas9 or D10A-Cas9 was transfected on day 1, and then, the BamHI oligo was co-transfected with sgRNA(s). Cells were collected for analysis three days after sgRNA transfection or isolated clones after puromycin selection. As shown in FIG. 6A, BamHI oligo insertion was observed in both Cas9 and D10A-Cas9 cleavaged samples by the BamHI digestion of the PCR product at Rab11A locus. Cell clones were also isolated and checked the BamHI oligo insertion. As shown in FIG. 6B, BamHI oligo was detected in 4 of 6 clones in iPSCs, and 8 of 11 clones in U2OS cells. Second, we inserted the TagGFP2 fragment at the GAPDH gene locus. HEK293 cells were transfected with a self-replicative Cas9-TagRFP on day1, and then, PCR amplified GFP fragment, and sgRNA were co-transfected on day2. GFP positive cells were observed 3 days after sgRNA transfection (FIG. 6C). These data suggest that a self-replicative Cas9 works for DNA insertion for genome editing.

1. A self-replicating RNA vector comprising a sequence encoding a plurality of non-structural replication complex proteins from an alphavirus and a sequence encoding a CRISPR protein.

2. The self-replicating RNA vector of claim 1, wherein the CRISPR protein is a type II Cas9 protein, a type V Cas12 protein, a type VI Cas13 protein, a CasX protein, or a CasY protein.

3. The self-replicating RNA vector of claim 1, wherein the CRISPR protein is Streptococcus pyogenes Cas9, Francisella novicida Cas9, Staphylococcus aureus Cas9, Streptococcus thermophilus Cas9, Streptococcus pasteurianus Cas9, Campylobacter jejuni Cas9, Neisseria meningitis Cas9, Neisseria cinerea Cas9, Francisella novicida Cas12, Acidaminococcus sp. Cas12, Lachnospiraceae bacterium ND2006 Cas12, Leptotrichia wadei Cas13a, Leptotrichia shahii Cas13a, Prevotella sp. P5-125 Cas13, or Ruminococcus flavefaciens Cas13d.

4. The self-replicating RNA vector of claim 3, wherein the CRISPR protein is Streptococcus pyogenes Cas9 or Staphylococcus aureus Cas9.

5. The self-replicating RNA vector of claim 1, wherein the sequence encoding the CRISPR protein comprises at least one nucleotide insertion, deletion, and/or substitution such that the CRISPR protein has altered catalytic activity, improved target site specificity, and/or decreased off-target effects.

6. The self-replicating RNA vector of claim 1, wherein the CRISPR protein is a nuclease, a nickase, or is devoid of cleavage activity.

7. The self-replicating RNA vector of claim 1, wherein the CRISPR protein is linked to at least one nuclear localization signal.

8. The self-replicating RNA vector of claim 1, wherein the CRISPR protein is linked to at least one fluorescent protein, at least one chromatin modulating motif, at least one functional domain, or combination thereof.

9. The self-replicating RNA vector of claim 8, wherein the at least one functional domain is an epigenetic modification domain, a transcriptional activation domain, or a transcriptional repressor domain.

10. The self-replicating RNA vector of claim 1, wherein the sequence encoding the CRISPR protein is codon optimized for expression in a human cell.

11. The self-replicating RNA vector of claim 1, wherein the alphavirus is Aura virus, Babanki virus, Barmah Forest virus, Bebaru virus, Buggy Creek virus, Chikungunya virus, Eastern equine encephalitis virus, Everglades virus, Fort Morgan virus, Getah virus, Highlands J virus, Kyzylagach virus, Mayaro virus, Middelburg virus, Mucambo virus, Ndumu virus Pixuna virus, O′nyong-nyong virus, Ross River virus, Sagiyama virus, Semliki Forest virus, Sindbis virus, Una virus, Venezuelan equine encephalitis virus, Western equine encephalitis virus, or Whataroa virus.

12. The self-replicating RNA vector of claim 11, wherein the alphavirus is a Venezuelan equine encephalitis virus.

13. The self-replicating RNA vector of claim 1, wherein the vector further comprises a sequence encoding at least one selectable marker.

14. The self-replicating RNA vector of claim 1, wherein the vector further comprises a sequence encoding an E3L protein.

15. The self-replicating RNA vector of claim 1, wherein the vector is based on a modified Venezuelan equine encephalitis (VEE) virus and comprises from 5′ to 3′: a 5′ cap, a 5′ UTR, the sequence encoding the plurality of non-structural replication complex proteins encodes four non-structural replication complex proteins from a VEE virus, a promoter, the sequence encoding the CRISPR protein, an optional IRES, an optional sequence encoding an E3L protein, an optional IRES, an optional sequence encoding a selectable marker, an alphavirus 3′ UTR, and a poly A tail.

16. A complex comprising the self-replicating RNA vector of claim 1, and at least one guide RNA that is engineered to complex with the CRISPR protein coded by the self-replicating RNA vector.

17. A eukaryotic cell or cell line comprising the self-replicating RNA vector of claim 1.

18. The eukaryotic cell or cell line of claim 17, further comprising at least one guide RNA that is engineered to complex with the CRISPR protein coded by the self-replicating RNA vector.

19. A plasmid vector encoding the self-replicating RNA vector as specified in claim 1.

20. The plasmid vector of claim 16, further comprising a T7 or SP6 promoter for in vitro transcription.

21. A method for targeted genome editing, the method comprising introducing into a eukaryotic cell the self-replicating RNA vector of claim 1 and at least one guide RNA that is engineered to complex with the CRISPR protein coded by the self-replicating RNA vector.

22. The method of claim 21, further comprising introducing into the cell at least one donor polynucleotide.

23. The method of claim 21, wherein the eukaryotic cell is a human cell.
